(19) United States
(12) Patent Application Publication
(10) Pub. No.: US 2008/0220389 A1
(43) Pub. Date: Sep. 11, 2008

(54) Circulating cooling structure for optical curing apparatus

(76) Inventor: Shu-Lung Wang, Taipei Hsien (TW)

Correspondence Address: Joe McKinney Muncy, PO Box 1364, Fairfax, VA, 22038-1364, US

(21) Appl. No.: 11/714,771

(22) Filed: Mar. 7, 2007

(51) Int. Cl. A61N 5/06

(52) U.S. Cl. 433/29

(57) ABSTRACT

A circulating cooling structure for optical curing apparatus which emits light of a selected wavelength to cure dental material such as a filler of teeth includes a casing and a heating element located in the casing which has an air inlet at one side where the heating element is located in a corresponding manner to receive air intake and an air outlet to allow the intake air to pass through the heating element and be discharged outside, thereby to provide an effective cooling means. When the optical curing apparatus is in use the air inlet is located at a lower side while the air outlet is located at a higher side, thus heated air flows upwards due to convection and external air can pass through the heating element to carry away a portion of heat energy and be discharged outside to achieve cooling effect.

### FIELD OF THE INVENTION

The present invention relates to an optical curing apparatus used in dentistry and particularly to a circulating cooling structure for optical curing apparatus.

### BACKGROUND OF THE INVENTION

The conventional method for mending tooth in the dentistry is filling a composite resin in a tooth cavity and projecting light to cure the resin.

The traditional “Curing apparatus using halogen light” mainly has an optical filter which allows blue light between 400 and 500 nm in the spectrum to pass through to cure the resin. But the halogen light easily generates high temperature that irritates tooth marrow and inflicts pain. This results in more physical pain and psychological uneasiness to patients.

In recent years LED (light emitting diode) curing light has been developed. LED is a semiconductor optical light that directly transforms electric energy to optical energy. By applying a voltage at a positive electrode and a negative electrode in a semiconductor, when current passes through electrons and electronic holes are coupled, residual energy is released in the form of light.

However in the conventional optical curing apparatus, whether adopted halogen or LED, the lighting element generates high heat when in use for a period of time. To remedy this problem the conventional technique usually connects a cooling element to the lighting element, or has an air fan directing the cooling element to channel air to the cooling element and lighting element to disperse heat. But resorting the air fan alone often cannot lower the temperature as desired. In the conventional structure the cooling element is directly held in a plastic casing. The heat energy of the cooling element is directly transferred to the plastic casing. The plastic casing receives heat and becomes brittle, or even melts and deforms. This could result in a serious consequence.

### SUMMARY OF THE INVENTION

The primary object of the present invention is to provide a circulating cooling structure for optical curing apparatus. When the optical curing apparatus of the invention is in use, external air enters a casing through an air inlet. Through air convection characteristics, the entering air passes through a heating element inside and carries a portion of heat energy and discharges outside to disperse heat more effectively.

To achieve the foregoing object, in an embodiment of the invention the optical curing apparatus has a casing and a heating element (which may be a lighting element and a cooling element thereof) located in the casing. The casing has an air inlet formed at one side where the heating element is located in a corresponding manner to receive air intake, and an air outlet to allow the intake air to flow through the heating element and be discharged through an air outlet. Thereby an effective cooling effect can be achieved.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of an embodiment of the invention.

FIG. 2 is an exploded view of an embodiment of the invention.

FIG. 3 is a schematic view of an embodiment of the invention in a use condition.

### DETAILED DESCRIPTION OF THE PREFERRED EMBODIMENT

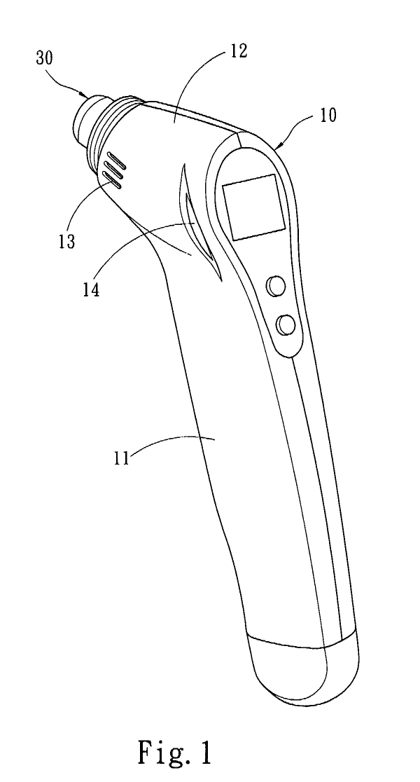

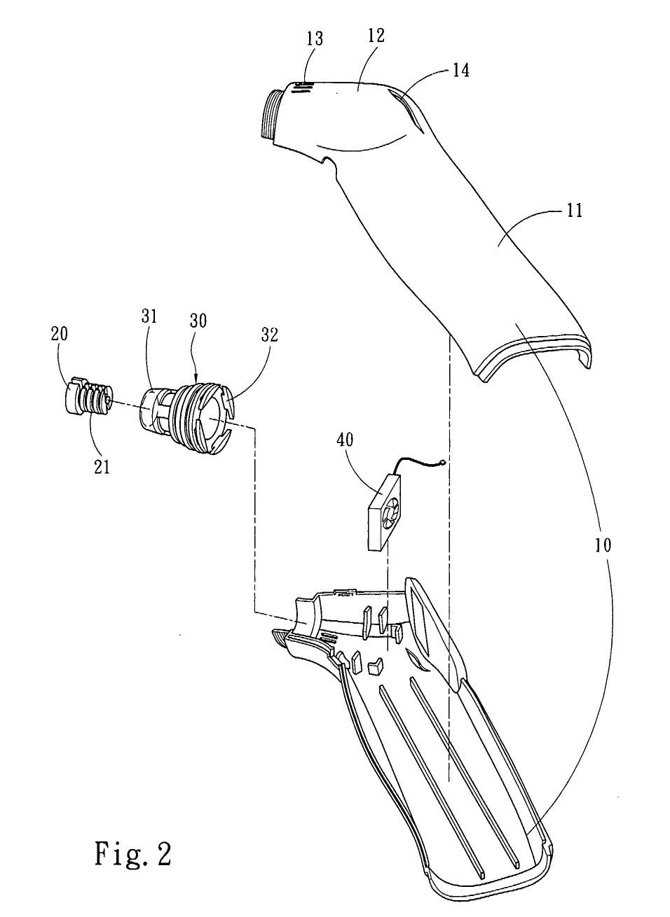

Please refer to FIG. 1 for an embodiment of the invention. The circulating cooling structure for optical curing apparatus of the invention has a casing 10 which has a handgrip 11 and an emission end 12 connecting to the handgrip 11. The emission end 12 and the handgrip 11 form an included angle between them. The emission end 12 holds a lighting element 20 inside (also referring to FIG. 2). The lighting element 20 may be a LED to emit light at a selected wavelength to cure dental material such as a filler of teeth.

When in use, the lighting element 20 generates high temperature. Hence the casing 10 also contains a cooling element 30 connecting to the lighting element 20 to rapidly receive heat energy transferred from the lighting element 20. Thus the cooling element 30 becomes a heating element. The cooling element 30 is hollow and has a front end 31 connecting to the lighting element 20, and a rear end 32 with an outer diameter greater than the front end 31 such that it becomes substantially a conical structure to accelerate transfer and dissipation of the heat energy. Moreover, the front end 31 and the lighting element 20 may also have respectively an internal screw thread (not shown in the drawings) and an external screw thread 21 that correspond to each other for fastening with each other. Thereby the lighting element 20 can be screwed in the cooling element 30. When the lighting element 20 is used for a long period of time or malfunctions, the lighting element 20 can be removed and separated from the cooling element 30 for replacement without disassembling the casing 10. Thus repairs and maintenance are more convenient.

To facilitate heat dissipation, the casing 10 may further include an air fan 40 which is directed to the cooling element 30 and the lighting element 20 to blow air to the cooling element 30 to lower the temperature. In addition, through a circuit board (not shown in the drawings) the air fan 40 may be set to operate and rotate only when the interior temperature is lower than a preset temperature (such as between 45 and 50 degrees Celsius). Aside from the air fan 40 to facilitate heat dissipation, the casing 10 also has an air inlet 13 at one side where the cooling element 30 is located in a corresponding manner to receive air intake, and an air outlet 14 to allow the intake air to pass through the cooling element 30 and be discharged outside.

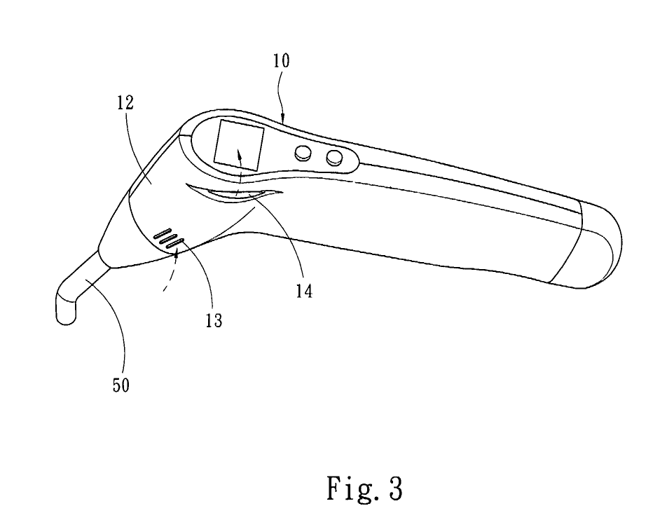

Refer to FIG. 3 for the embodiment of the invention in a use condition. The invention provides an effective heat dissipation means. When the optical curing apparatus is in use (an extra extension duct 50 may be provided and coupled), according to the positions of the patient and dentist, the air inlet 13 is positioned naturally at a lower side while the air outlet 14 is located at a higher side. Heated air flows upwards due to convection so that external air passes through the lighting element 20 and cooling element 30 to carry away a portion of heat energy outside. When the interior temperature does not reach a preset temperature, the air fan 40 does not start. Heat dissipation can be achieved through the air inlet 13 and air outlet 14 previously discussed so that the work load of the air fan 40 can be reduced and the life span is longer. When the interior temperature reaches the preset level, the air fan 40 starts operation to disperse heat as desired.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A circulating cooling structure for optical curing apparatus to generate light of a selected wavelength to cure dental material, comprising:
a casing and a heating element located in the casing, the casing having an air inlet at one side where the heating element is located in a corresponding manner to receive air intake and an air outlet to allow the intake air to pass through the heating element and be discharged outside.

2. The circulating cooling structure for optical curing apparatus of claim 1, wherein the casing has a handgrip and an emission end connecting to the casing, the emission end and the handgrip forming an included angle.

3. The circulating cooling structure for optical curing apparatus of claim 2, wherein the emission end holds a lighting element to generate the light.

4. The circulating cooling structure for optical curing apparatus of claim 3, wherein the lighting element is a light emitting diode.

5. The circulating cooling structure for optical curing apparatus of claim 3, wherein the casing has a cooling element connecting to the lighting element to become the heating element.

6. The circulating cooling structure for optical curing apparatus of claim 5, wherein the cooling element is hollow and has a front end connecting to the lighting element and a rear end formed at an outer diameter greater than the front end.

7. The circulating cooling structure for optical curing apparatus of claim 6, wherein the front end and the lighting element have respectively an internal screw thread and an external screw thread that correspond to each other for fastening with each other.
